(19) United States
(12) Patent Application Publication
(10) Pub. No.: US 2022/0117940 A1
(43) Pub. Date: Apr. 21, 2022

(54) COMPOSITION FOR INCREASING PREGNANCY RATE OF RUMINANTS AND PREPARATION METHOD AND APPLICATION THEREOF

(71) Applicant: CHINA AGRICULTURAL UNIVERSITY, Beijing (CN)

(72) Inventors: Guoshi LIU, Beijing (CN); Hao WU, Beijing (CN); Lu ZHANG, Beijing (CN); Yongqiang LU, Beijing (CN); Tiankun WANG, Beijing (CN); Jiangpeng GUO, Beijing (CN); Songyang YAO, Beijing (CN); Shengyu GUAN, Beijing (CN); Pengyun JI, Beijing (CN)

(21) Appl. No.: 17/502,761

(22) Filed: Oct. 15, 2021

(30) Foreign Application Priority Data
Oct. 16, 2020 (CN) 202011112550.4

(51) Int. Cl. A61K 31/4045; A61K 9/00

(52) U.S. Cl. CPC A61K 31/4045; A61K 9/0019

(57) ABSTRACT

Disclosed is a composition for increasing pregnancy rate in ruminants exposed to ovsynch and timed artificial insemination, and a preparation method and application thereof. The effective component of the composition is melatonin with a concentration of 10-20 mg/mL. The present disclosure proves through animal experiments that the use of the composition of the present disclosure in combination with the last injection of GnRH in an ovsynch and timed artificial insemination protocol for a ruminant can regulate the secretion of reproductive hormones in an animal body, increase the levels of luteinizing hormone (LH) and progesterone in pregnant females, and improve ovum quality and facilitate ovulation, thus increasing the pregnancy rate in ruminants exposed to ovsynch and timed artificial insemination.

### FIELD OF TECHNOLOGY

The present disclosure relates to the field of animal husbandry and veterinary medicine, particularly to a composition for increasing pregnancy rate in ruminants exposed to ovsynch and timed artificial insemination, a preparation method and application thereof.

### BACKGROUND

In recent years, the gradual increase of yield in the dairy industry has led to a declining pregnancy rate in multipara cows exposed to artificial insemination and longer calving intervals. This is seriously affecting the economic efficiency. How to improve the reproductive efficiency of dairy cattle has become an urgent problem to be solved for the development of the industry. Ovsynch timed artificial insemination (TAI) refers to a technology in which a group of cows comes into heat and ovulates in the same period, and then is artificially inseminated at a fixed time. The technology is effective and efficient, and has been applied in many large ranches. Classic ovsynch TAI technology comprises Ovsynch protocol (GnRH-PG-GnRH), Presynch protocol (PG-PG-GnRH-PG-GnRH), Richard Pursley protocol, and the like. However, in practice, these protocols have strict requirements and take long time. In addition, it is required to use a lot of drugs such as GnRH (Gonadotropin-Releasing Hormone) and its analogues, which can lead to corresponding damages, such as inhibition of ovulation or gonadal atrophy to dairy cows. Therefore, it is urgent to optimize the ovsynch TAI protocol of the dairy cows to further meet the need of production.

Melatonin (MT), chemically known as N-acetyl-5-methoxytryptamine, is a hormone primarily secreted by the pineal gland. Melatonin has strong antioxidant properties and plays an important regulatory role in animal reproduction. During the in vitro maturation of oocytes, the addition of melatonin can promote the maturation of oocytes and enhance the potential of embryonic development after external fertilization or parthenogenetic activation. This suggests that melatonin should be directly involved in the regulation of oocyte maturation. In vivo studies have found that preovulatory in vivo luteinizing hormone (LH) peaks can cause a sharp increase in the expression level of type I (MT1) melatonin receptor in ovarian granulosa cells, with extremely remarkable increase in the expression level of melatonin synthase AANAT in cumulus cells, which leads to an increase in the level of melatonin in follicular fluid. This suggests that melatonin is a downstream signaling molecule of LH, acting through the MT1 receptor. It can significantly increase the level of progesterone in mouse serum and upregulate the expression of corpus luteum marker genes Akr1c18 and Cyp11a1 by injecting melatonin at a dose of 5 mg/kg before the MT1 receptor peak.

The low pregnancy rate of multipara cows exposed to artificial insemination is one of the main factors affecting the efficiency of dairy production. Large amounts of reactive oxygen species (ROS) are produced during normal ovulation in cows, which seriously affect the quality of oocytes.

Therefore, it is necessary to find an effective and feasible method to remove the large amounts of ROS produced during ovulation and improve the quality of oocytes, so as to increase the pregnancy rate in dairy cows exposed to artificial insemination.

### SUMMARY

In order to solve the problems in the prior art, the object of the present disclosure is to provide a composition for increasing pregnancy rate and reproduction rate in ruminants exposed to ovsynch timed artificial insemination (TAI), and a preparation method and application thereof.

In order to realize the object of the present disclosure, the technical solutions of the present disclosure are as follows:

In the first aspect of the present disclosure, provided is a composition for increasing pregnancy and reproduction rates in ruminants, which comprises an active ingredient containing melatonin. Preferably, the active ingredient of the composition may be melatonin.

Further, the composition may comprise melatonin at a concentration of 10-20 mg/mL, equivalent to 4.3×10⁻²-8.6×10⁻² mol/L.

According to a preferable embodiment, the composition may comprise melatonin at a concentration of 15 mg/mL.

Further, the composition may further comprise anhydrous ethanol and physiological saline.

Preferably, the volume ratio of the anhydrous ethanol and physiological saline may be in a range of (1-3):(1-2), and more preferably 3:2.

According to a preferable embodiment, the purity of anhydrous ethanol is of 99% or more; the physiological saline is 0.9% physiological saline.

According to a preferable embodiment, the volume ratio of anhydrous ethanol and 0.9% physiological saline in the composition may be in a range of (1-3):(1-2), preferably 3:2.

Further, the composition may be in a form of an injection.

In the second aspect of the present disclosure, provided is a preparation method of the composition, which comprises the following steps: dissolving melatonin in anhydrous ethanol and then adding physiological saline, the steps are performed in dark.

According to a preferable embodiment, the present disclosure further provides a preparation method of the composition, which comprises the following steps: dissolving melatonin in anhydrous ethanol and then adding 0.9% physiological saline, the steps are performed in dark.

In the third aspect of the present disclosure, provided is use of the composition or a composition prepared by the above method in increasing pregnancy rate in ruminants exposed to ovsynch and timed artificial insemination (TAI).

According to a preferable embodiment, for the above use of the present disclosure, during the process of GnRH-PG-GnRH or PG-PG-GnRH-PG-GnRH of ovsynch TAI in ruminants, the composition of the present disclosure or a composition prepared by the above method is administrated while injecting a last dose of GnRH, and 24 to 48 hours later, insemination is performed. Preferably, the insemination may be performed 24 hours after the administration of the composition.

Preferably, the composition may be administrated at a dose of 7.5×10⁻²-11.25×10⁻² mg/kg ruminant, which is calculated by melatonin.

In the fourth aspect of the present disclosure, provided is use of the composition or a composition prepared by the above method in increasing reproduction rate in ruminants exposed to ovsynch and timed artificial insemination (TAI).

According to a preferable embodiment, for all of the above uses of the present disclosure, during the process of GnRH-PG-GnRH or PG-PG-GnRH-PG-GnRH of ovsynch TAI in ruminants, the composition of the present disclosure or a composition prepared by the above method is administrated while injecting a last dose of GnRH, and 24 to 48 hours later, insemination is performed. Preferably, the insemination may be performed 24 hours after the administration of the composition.

Preferably, the composition may be administrated at a dose of 7.5×10⁻²-11.25×10⁻² mg/kg ruminant, which is calculated by melatonin.

In the fifth aspect, the composition of the present disclosure or a composition prepared by the method of the present disclosure is administered in a form of an injection.

Further preferably, the composition is administrated by subcutaneous injection.

According to a preferable embodiment, during the process of ovsynch TAI in ruminants, the composition of the present disclosure or a composition prepared by the above method is administered through subcutaneous injection while administrating the last injection of GnRH, in order to increase the pregnancy and/or reproduction rates in ruminants.

According to a preferable embodiment, the composition may be administrated at a dose of 7.5×10⁻²-11.25×10⁻² mg/kg ruminant, preferably 7.5×10⁻²-9.4×10⁻² mg/kg, and more preferably 7.5×10⁻² mg/kg, which is calculated by melatonin.

Further, the composition may be administrated at a dosing of 2-3 mL, preferably of 2 mL.

The ruminants of the present disclosure are selected from a group consisting of dairy cows, yellow cattle, water buffalo, goats, domestic sheep, camels, alpacas, antelopes and gazelles.

The technical solutions provided by the present disclosure at least comprise the following beneficial effect.

It is unexpectedly found in the present disclosure that melatonin can be used for increasing the pregnancy rate in dairy cows exposed to the ovsynch TAI. The pregnancy rate of cows can be increased by dissolving melatonin in anhydrous ethanol and physiological saline to give an injection, and injecting the injection of melatonin of the present disclosure in combination with GnRH injection for the ovsynch TAI of the dairy cows. It can provide a beneficial reference for further research in order to improve the reproductive efficiency of the cows.

With animal experiments, it is found in the present disclosure, for example in a preferred embodiment, a preparation comprising melatonin of 15 mg/mL can increase the pregnancy rate by 11% in cows exposed to the ovsynch TAI. This is of great significance and economic value for the dairy industry.

In the present disclosure, the subcutaneous injection of melatonin together with the injection of GnRH in an ovsynch TAI protocol for the dairy cows can effectively reduce the damage of ROS on ovum quality, promote ovulation and sperm-ovum fusion to achieve fertilization, and form functional corpus luteum and progesterone secretion after ovulation.

Moreover, it is unexpectedly found in the present disclosure through animal experiments that the levels of LH and E₂ (estradiol) in cows 8 hours after injection of GnRH can be remarkably increased by the subcutaneous injection of melatonin. Such changes in hormone has a remarkable promotion effect in increasing the pregnancy rate of cows.

According to the present disclosure, the subcutaneous injection of melatonin can be used for removing a large amount of ROS during ovulation, improving oocyte quality, promoting luteinization of follicular granulosa cells, and forming a functional corpus luteum after ovulation. Meanwhile, melatonin can also regulate the secretion of other reproductive hormones, which in turn regulates and promotes oocyte development and sperm-ovum fusion, resulting in the increase of the pregnancy rate in dairy cows. Furthermore, the technical system provided by the present disclosure is simple, practical and operational.

### BRIEF DESCRIPTION OF THE DRAWINGS

FIG. 1 shows an exemplary diagram of administrate to Holstein cows exposed to ovsynch TAI (PG-PG-GnRH-PG-GnRH protocol) with a melatonin injection in Experimental Example 1 of the present disclosure.

FIG. 2 shows a diagram of changes in the levels of MT, FSH (Follicle Stimulating Hormone) and LH after the injection of melatonin into Holstein cows exposed to ovsynch TAI in Experimental Example 1 of the present disclosure.

Changes in the levels of MT, FSH and LH after the injection of MT are as follows:

A: changes in MT levels: (4.3465±0.5286 VS 2.986±0.3801), wherein n=20 (unit: cows);

B: changes in FSH levels: (2.482±0.24629 VS 2.2096±0.11549), wherein n=25 (unit: cows); and

C: changes in LH levels: (5.022±0.23237 VS 4.2068±0.32362), wherein n=25 (unit: cows).

All data are expressed as mean values±standard error, and the symbol “*” represents significant differences between groups.

FIG. 3 shows a diagram of changes in the levels of P (progesterone) and E₂ after the injection of melatonin into Holstein cows exposed to ovsynch TAI in Experimental Example 1 of the present disclosure.

Changes in levels of P and E₂ after the injection of MT are as follows:

A: changes in P levels: (0.1825±0.0235 VS 0.1215±0.00898), wherein n=20 (unit: cows); and

B: changes in E₂ levels: (11.678±0.88344 VS 9.2776±0.60414), wherein n=25 (unit: cows).

All data are expressed as mean value±standard error, and the symbol “*” represents significant differences between groups.

### DESCRIPTION OF THE EMBODIMENTS

Examples are provided hereinafter to demonstrate the present disclosure, but not intend to be used to limit the scope of the present disclosure.

All the reagents and materials used in the following examples are commercially available.

The purity of melatonin is required to reach 99.9%.

### Example 1. Preparation of a Melatonin Injection

Raw materials: melatonin and anhydrous ethanol.

Preparation process: accurately weigh 1.5 g of melatonin and dissolve with anhydrous ethanol in a 100-mL volumetric flask, stir well, then add 0.9% physiological saline and stir well, and volume to 100 mL (anhydrous ethanol: physiological saline=3:2) to give a solution of 15 mg/mL which was dispensed in 2-mL doses for injection. The whole process was done in a dark room.

### Example 2. A Technical System for Ovsynch and Timed Artificial Insemination (TAI) in Dairy Cows in Combination with Melatonin Injection

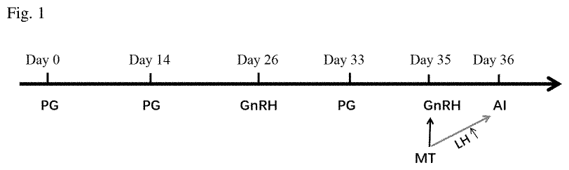

A melatonin injection of 2-3 mL was injected in combination with the injection of GnRH of ovsynch TAI in cows (as shown in FIG. 1), with B-scan pregnancy tests on Days 32-35, or pregnancy tests by recto-vaginal technique on Day 60. The pregnancy rates were calculated.

### Example 3. Analysis of the Regulation Function of Subcutaneous Injection of Melatonin into Cows Exposed to Ovsynch on Reproductive Hormones

The process is substantially same as that of Example 2, except that blood was collected at each stage from ovsynch TAI to pregnancy tests, in order to determine the changes in relevant reproductive hormones in ovulating cows.

### Experimental Example 1

1. Patterns of Changes of MT in Cows after Ovsynch TAI

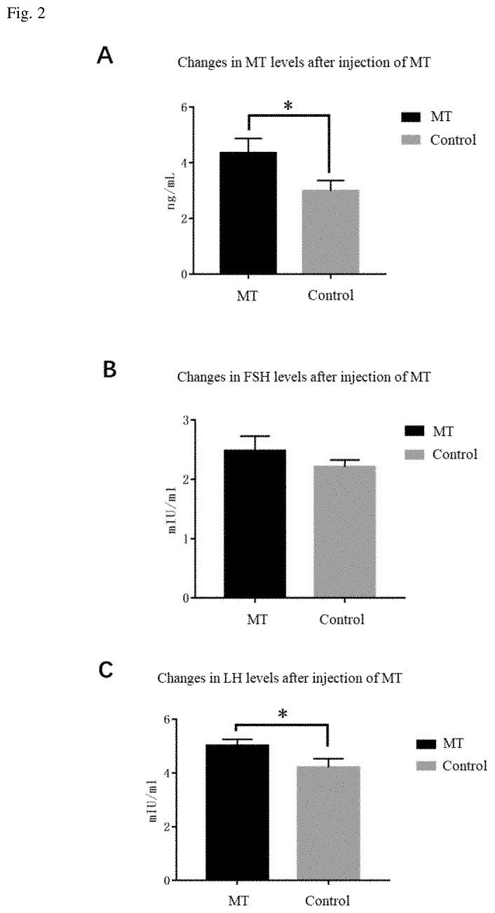

Twenty-five multipara (1-2 pregnancies) Holstein cows with a mean body weight of about 400 Kg were adopted, and were fed, drunk and milked normally. Blood of the cows was collected from ovsynch to TAI, until pregnancy tests. The concentrations of serum MT were determined by liquid chromatography and the results are shown in FIG. 2.

As can be seen from FIG. 2, the melatonin levels were remarkably increased (4.35±0.53 VS 2.99±0.38, P<0.05) in blood when inject melatonin, while inject the last dose of GnRH into Holstein cows exposed to a PG-PG-GnRH-PG-GnRH protocol of ovsynch TAI.

2. Patterns of Changes of Other Reproductive Hormones in Cows after Ovsynch TAI

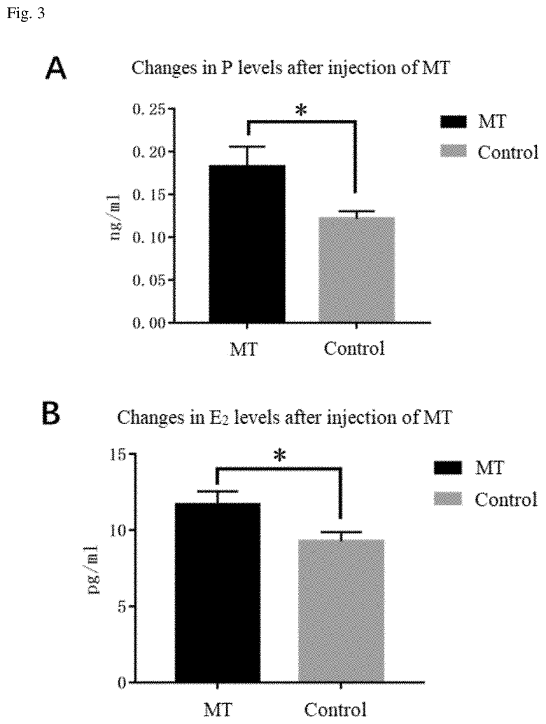

Twenty-five multipara (1-2 pregnancies) Holstein cows were fed, drunk and milked normally. Blood of the cows was collected from ovsynch to TAI, until pregnancy tests. The levels of other reproductive hormones in serum were determined and the results are shown in FIGS. 2 and 3.

As can be seen from FIGS. 2 and 3, both of the LH (5.02±0.23 VS 4.21±0.32, P<0.05) and E₂ (11.68±0.88 VS 9.28±0.60, P<0.05) levels in blood were increased remarkably after inject melatonin while inject the last dose of GnRH into Holstein cows exposed to ovsynch TAI. In contrast, there was no significant change in FSH levels (2.48±0.25 VS 2.21±0.12, P>0.05). On Day 30 of pregnancy, the progesterone levels in pregnant cows were increased remarkably (0.18±0.02 VS 0.12±0.01, P<0.05).

3. Melatonin Injection in Combination with the Last Injection of GnRH into Cows Exposed to Ovsynch TAI

2 mL of the injection prepared in Example 1 was administrated together with the last injection of GnRH to multipara Holstein cows (1-3 pregnancies) exposed to postpartum ovsynch TAI. In this process, a randomized administration approach was used, for example administer to every other cow in which the first cow was administrated, the second was not administrated, the third was administrated, and so on.

4. Detection of the Pregnancy Rates in Cows

50 to 60 days after the insemination, the pregnancy tests were performed on the Holstein cows by means of rectal examination. Alternatively, 32 days after the insemination, the pregnancy rates were calculated by means of B-scan ultrasonography and pregnancy detection kits. It was found that the pregnancy rates of cows were significantly increased by more than 36.42% (9.68/26.58) by injecting the preparation according to the present disclosure into multipara cows during the ovsynch TAI process (see Table 1). The pregnancy rates were significantly increased by more than 55.62% (14.60/26.25) by injecting melatonin during natural ovulation (see Table 2). The pregnancy rates of the cows exposed to TAI or natural ovulation were recorded, and the results showed that injection of melatonin significantly increased the pregnancy rates of the cows by more than 44.85% (11.85/26.42) (see Table 3).

TABLE 1
Effect of the injection of melatonin on the pregnancy
rates of Holstein cows exposed to TAI.

| | | Total | Number of | | | |
|---|---|---|---|---|---|---|
| Ovulation | | number of | pregnant | Pregnancy | Number of | Calving |
| method | Group | cows | cows | rate | calves | rate |
| TAI | Melatonin | 91 | 33 | 36.26%^{a} | 23 | 25.27%^{a} |
| | Control | 79 | 21 | 26.58%^{b} | 15 | 18.99%^{b} |

Note:
in the same column, if the superscript lowercase letters are different, it indicates there is a significant difference between the groups (P < 0.05); if the superscript lowercase letters are the same, there is no significant difference between the groups (P > 0.05).

TABLE 2
Effect of the injection of melatonin on the pregnancy
rates of Holstein cows exposed to natural ovulation.

| | | Total | Number of | | | |
|---|---|---|---|---|---|---|
| Ovulation | | number of | pregnant | Pregnancy | Number of | Calving |
| method | Group | cows | cows | rate | calves | rate |
| Natural | Melatonin | 71 | 29 | 40.85%^{A} | 21 | 29.58%^{A} |
| ovulation | Control | 80 | 21 | 26.25%^{B} | 15 | 18.75%^{B} |

Note:
in the same column, if the superscript capital letters are different, it indicates there is a significant difference between the groups (P < 0.05); if the superscript capital letters are the same, it indicates there is no significant difference between the groups (P > 0.05).

TABLE 3
Effect of the injection of melatonin on the pregnancy
rates of first insemination in cows.

| | Total number | Number of | | | |
|---|---|---|---|---|---|
| | of first | pregnant | Pregnancy rate of | Number of | Calving |
| Group | insemination | cows | first insemination | calves | rate |
| MT group | 162 | 62 | 38.27%^{a} | 44 | 27.16%^{a} |
| Control group | 159 | 42 | 26.42%^{b} | 30 | 18.87%^{b} |

Note:
in the same column, if the superscript lowercase letters are different, it indicates there is a significant difference between the groups (P < 0.05); if the superscript lowercase letters are the same, there is no significant difference between the groups (P > 0.05).

The above results demonstrate that the in vivo concentration of MT is remarkably increased after injecting melatonin at the time of injecting the last dose of GnRH into Holstein cows exposed to ovsynch TAI. Meanwhile, the in vivo levels of LH and E₂ were increased remarkably 8 hours after the injection of GnRH, resulted in enhanced oestrus and ovulation of the cows. GnRH can promote the secretion of FSH and LH. LH can promote the ovulation of mature follicles. The increased LH concentration can thus promote more mature follicles to be ovulated, and improve ovulation efficiency. Meanwhile, melatonin can remove ROS produced during the ovulation, and thus reduce the oxidative stress damage. Melatonin can promote the formation of corpus luteum and further increase the progesterone levels in pregnant cows, resulting in significantly increased pregnancy rate of first insemination.

Although the present invention has been described in detail with general description and specific embodiments, it would be obvious for those skilled in the art that some modifications or improvements can be made on the basis of the present invention. Therefore, these modifications or improvements made without deviating from the spirit of the present invention are within the claimed scope of protection of the present invention.

What is claimed is:

1. A composition for increasing pregnancy and reproduction rates in ruminants, comprising an active ingredient containing melatonin.

2. The composition according to claim 1, wherein the active ingredient of the composition is melatonin.

3. The composition according to claim 1, wherein the composition comprises melatonin at a concentration of 10-20 mg/mL.

4. The composition according to claim 1, further comprising anhydrous ethanol and physiological saline; and the volume ratio of anhydrous ethanol and physiological saline is in a range of (1-3):(1-2).

5. The composition according to claim 4, wherein the volume ratio of absolute ethanol to physiological saline is in a range of 3:2.

6. The composition according to claim 1, wherein the composition is in a form of an injection.

7. A method for preparing the composition of claim 1, comprising the following steps: dissolving melatonin in anhydrous ethanol and then adding physiological saline, wherein the steps are performed in dark.

8. The method according to claim 7, wherein the active ingredient of the composition is melatonin.

9. The method according to claim 7, wherein the composition comprises melatonin at a concentration of 1 to 20 mg/mL.

10. The method according to claim 7, wherein the volume ratio of absolute ethanol to physiological saline is in a range of (1-3):(1-2).

11. The method according to claim 10, wherein the volume ratio of absolute ethanol to physiological saline is in a range of 3:2.

12. The method according to claim 7, wherein the composition is prepared as a form of injection.

13. A method for increasing pregnancy rate and/or reproduction rate in ruminants exposed to ovsynch and timed artificial insemination (TAI), comprising the following steps: during the process of GnRH-PG-GnRH or PG-PG-GnRH-PG-GnRH of the TAI in ruminants, administrate a composition comprising an active ingredient containing melatonin, while injecting a last dose of GnRH; and, 24 to 48 hours later, perform insemination.

14. The method according to claim 13, wherein the composition is administrated at a dose of 7.5×10⁻²-11.25×10⁻² mg/kg ruminant, which is calculated by melatonin.

15. The method according to claim 13, wherein the active ingredient of the composition is melatonin.

16. The method according to claim 13, wherein the composition comprises melatonin at a concentration of 1 to 20 mg/mL.

17. The method according to claim 13, wherein the composition further comprises absolute ethanol and physiological saline; the volume ratio of absolute ethanol to physiological saline is in a range of (1-3):(1-2).

18. The method according to claim 17, wherein the volume ratio of absolute ethanol to physiological saline is in a range of 3:2.
